(19) United States
(12) Patent Application Publication
(10) Pub. No.: US 2006/0263824 A1
(43) Pub. Date: Nov. 23, 2006

(54) Method for the use of information derived by the application of a probability law suitable for chiral selection and methods and systems employing same

(76) Inventors: Doron Lancet, Rehovot (IL); Ran Kafri, Nes Ziona (IL)

Correspondence Address: FAY, SHARPE, FAGAN, MINNICH & MCKEE, LLP, 1100 SUPERIOR AVENUE, SEVENTH FLOOR, CLEVELAND, OH, 44114, US

(21) Appl. No.: 10/547,963

(22) PCT Filed: Mar. 3, 2004
(86) PCT No.: PCT/IB04/00561

§ 371 Date: Jun. 13, 2006

Related U.S. Application Data
(60) Provisional application No. 60/450,687, filed on Mar. 3, 2003.

(51) Int. Cl. G01N 33/53

(52) U.S. Cl. 435/7.1

(57) ABSTRACT

A method of identifying molecules having enantion-discriminating regions comprising applying the chiral probability law disclosed herein to a plurality of molecules thereby identifying the molecules having enantio-discriminating regions.

The subject of this invention is a method for the use of information derived by the application of a statistical analysis and observations to enable, facilitate and optimize the determination of suitable selectors and selectands. This particularly holds for the selection of enantio-discriminating selectors and selectands. Covered by this invention are also technical systems to carry out the above mentioned methods.

Currently no method is known to systematically derive information for a given selector from the measurement of only some selectands regarding its ability to be enantio-discriminating or even determine a quantitative statistical measure thereof. By measuring the factor alpha (see definition in eq. 1) for only a few selectands it can be determined whether a compound constituting a selector column is effectively enantio-selective or not. By sampling the distribution of alpha for a small number of selectands and fitting the universal statistics as described below it is possible to quantitatively determine the enantio-separating ability of the selector. This issue is for the first time addressed in claim 1.

This information gain can further be increased by using additional information from relating the 3D-structure of the selector and a group of selectands (24), and relating it to a phenomenological model that explains the statistics and probability behavior of chiral recognition and selection. This issue is addressed in claim 2.

An example for a technical system carrying the measurement needed to determine all required data is given in FIG. 8. Details for the determination of the affinities in this special context are given e.g. in (Refs 24, 25). A device is set up which is able to separate (at least partially) the enantiomers (1) and subsequently measure the capacity factors which are related to the affinity values for both enantiomers (2). The measurement values are processed (3) and stored (4) in a processing unit (e.g. a computer) and afterwards compared to the already existing data in (4) to determine an estimation of the statistics. A general technical system solving the above mentioned tasks is claimed by claim 3.

In the daily practice it is desirable if for a given selector already after a few measurement with sample selectands an estimation on the numbers of experiment needed to be carried out can be given. By measuring samples a statistics is generated according to claim 1. It then can easily be calculated with high significance how many compounds a library of selectors must contain to a) either have at least a certain number of compounds above a predefined threshold of enantio-separating factor or b) have a certain number of selector column matrices or compounds within certain bounds. This issue is addressed in two variants in claim 4.

The setup for a technical system for carrying out this task is essentially already given in the context of claim 3. However, the analyzing methods carried out on the processing unit applied to the observed statistics differ from that claim and are realized in accordance with the methods described in the context of claim 4. A technical system solving the above mentioned tasks is given in claim 10.

It can also be desirable if the design of a chemical library is supported by measurement results about the enantio-discrimination of a small subsample of the library. Again a statistics from a subsample is estimated and based on that the necessary size of the library is determined. This issue is addressed in two variants in claim 5.

Since the technical realization of a device is from the hardware identical to the one given in claim 11 it is referred to here, the major difference being that the output of the processing unit are design requests for the library. A technical system solving the above mentioned tasks is given in claim 11.

In the daily practice it is desirable if for a given selector already after a few measurement with sample selectands of a chosen subset an estimation on the numbers of experiment needed to be carried out can be given. The same procedure as described in claim 4 is used only on a chosen subsample of the library. This issue is addressed in two variants in claim 6.

Since the technical realization of a device is from the hardware identical to the one given in claim 11 it is referred to it here, the major difference being the input to that system being only the chosen subset of the library. A technical system solving the above mentioned tasks is given in claim 12.

It can also be desirable if the design of chosen subset of a chemical library is supported by measurement results about the enantio-discrimination of a small subsample of the chosen subset of the library. The method to be applied for in this case is essentially the same as described in claim with the major difference being that it is now only applied to a chosen subset of the library to be designed. This issue is addressed in two variants in claim 7.

Since the technical realization of a device is from the hardware identical to the one given in claim 12 it is referred to it here, the major difference being the input to that system being only the chosen subset of the library. A technical system solving the above mentioned tasks is given in claim 13.

Based on the results carrying out the method described in claim 7 and e.g. realized by the system given in claim 3 a selection of the most suitable library for a given requirement profile is possible. This issue is addressed in claim 8.

Since the technical realization of a device is from the hardware identical to the one given in claim 12 it is referred to it here, the major difference being the input to that system is applied to sample of different libraries. The processing unit is then able to determine (with or without further limitations) the most suitable library for the given task. A technical system solving the above mentioned tasks is given in claim 14.

In an extension to claim 2 it is possible to enhance all methods and systems mentioned in claims 4-9 and 11-16 by incorporating at least part of the 3D-structure of either the selector or the selectand. This issue is addressed in claim 9.

A technical system solving the above mentioned tasks is given in claim 15.

Generally speaking, the String Model for Enantio-Discrimination or SMED model and algorithm provides a means of finding out what is the probability of a particular value of the efficacy of enantio-selection. This efficacy is denoted by a measurement of the physical constant alpha, which denotes the ratio of affinities (Capacity factors, see eq. 1) displayed by a selector (protein, chromatographic column and the like) towards the D and L (R and S) isomers of a chiral (asymmetric) compound. This model is similar to the Receptor Affinity Distribution (RAD) model that affords the knowledge of the probability of different values of ligand-receptor affinities. Like in RAD, the probability of a given alpha value may be interpreted as the inverse of the size of the repertoire needed to attain a particular molecular recognition feature.

The knowledge of the SMED distribution allows one to plan ahead, i.e. to guess what might be the number of different selectors needed to be screened before a value of alpha higher than a preset minimum is encountered. Analogously, it allows one to design and construct a combinatorial selector library of the appropriate size for the job at hand.

It is conceivable that different subtypes of selectors or of selectand will display a different shape of SMED-distribution. Thus future R&D could provide a fine-tuned SMED for different classes.

One of the point of strength of the SMED approach is “sampling”. In this realm, it is possible to do a limited number of experiments naturally leading to results with relatively low alpha values and obtain a trend that can then be used to extrapolate towards higher values of alpha.

The SMED string model core allows correlations to real structural data, thus enhancing the extrapolation capacity and this may be particularly useful for selectors or selectands of particular structural classes. Specifically, it is possible to relate the detailed molecular structure elements to the formal subsites in the SMED model and derive information as to why certain compounds are better selectors or selectands.

Very helpful in this context is the extension of the CHIRBASE database mentioned below to 3D-structural information as given in the CHIRSOURCE database (24) together with the additional information recorded therein. Molecular chirality is of central interest in biological studies because enantiomeric compounds, while indistinguishable by most inanimate systems, show profoundly different properties in biochemical environments. Enantio-selective separation methods, based on the differential recognition of two optical isomers by a chiral selector, have been amply documented. Yet, only a few attempts have been aimed at a systematic quantitative analysis of this molecular recognition phenomenon. Here we report a comprehensive data examination of enantio-separation measurements for over 75,000 chiral selector-selectand pairs from the chiral selection compendium CHIRBASE. The distribution of α=k′_{D}/k′_{L} values was found to follow a power law, equivalent to an exponential decay for chiral differential free-energies. A formalism is proposed to account for this observation on the basis of an extended Ogston three-point interaction model. Partially overlapping molecular interaction domains are analyzed in terms of a previously developed Poisson-distributed receptor affinity distribution model for ligand-receptor complementarity. The results suggest that chiral selection statistics may be interpreted in terms of more general concepts related to biomolecular recognition.

Introduction

The science of discrimination and separation of chiral compounds has been receiving enormous attention in the past decade due to its pharmaceutical importance and relevance to the more general topic of molecular recognition. Up to date, asymmetric reactions are rare and the most applicable tool for obtaining symmetry broken mixtures is chiral stationary phase (CSP) based separation technologies. Here, enantiomers are separated based on their differential recognition by an immobilized molecule in either gas or liquid chromatography. The precise mechanisms by which such discrimination takes place has been characterized for many model CSP/ligand pairs. It has been shown, for example, that the discrimination free-energy differences are typically 2-3 orders of magnitude lower that the binding energies of the ligand and stationary phase. Also, it has been shown that in most cases the location displaying maximum binding affinity towards the separated compounds is that which is most chiral discriminatory. Despite these studies, there are only few models attempting to provide a general understanding of enantio-selectivity [1-3]. Some of these include the well celebrated Ogston three point model, and Avnirs quantitative chirality measure providing a systematic approach linking chiral selectivity to molecular geometric features.

In accordance with the ever increasing accumulated data from various experimental scientific effort, a new line of disciplines has emerged whereby analysis of large experimental datasets give rise to new and sometimes unexpected understandings. This new mode of research has seen its implementations in the analysis of biological databases by what is called bio-informatics. In a similar approach it is possible to take large amount of chemical constants collected from different studies and through their analysis derive knowledge inaccessible otherwise. One example of such a chemo-informatics study is the effort to answer the long existing question of the affinity distribution first raised by Sips [4] in 1948. In the context of chromatographic separations, for example, an affinity distribution would constitute the frequency at which ligands with a particular retention time would occur in a random molecular collection. The derivation of affinity distributions from various data sources has been recently carried out and it was shown that different sources emerge with surprisingly similar distributions.

In another related example Roussel & Piras [5] have constructed a large and well organized database (CHIRBASE) listing details from thousands of chiral separations. Although CHIRBASE is constructed as a tool for deciding upon separation procedures for particular analytes it may also be used for broad statistical analysis of various aspects of chiral recognition. One such analysis [5] attempts to link various chemical descriptors of both selector and selectands to separation efficiencies and cluster separations according to these descriptors. In this report we attempt a less ambitions task of mapping the distribution of separation factors and resultant separation free-energy differences from the various CHIRBASE separations. We show that this seemingly naïve approach leads to unexpected and revealing information decrypting fundamental logic of chiral discrimination.

Methods

Chiral Chromatographic Separation Data Chromatographic enantiomer separation data were retrieved from the CHIRBASE database (1997-2000 ENSSPICAM, Marseille, France) [6, 7]. At the time of the study the data consisted of 72,076 chiral separation records for selectors with pairs of enantiomeric selectands. Each record includes molecular structures, molecular mass, chromatographic data and experimental conditions. The numerical value presented for all triads was the separation factor
α=k′₁/k′₂ (1)

where k′ᵢ are the capacity factors for the two optical isomers, relative to a given chiral stationary phase. In addition, for a large majority of the records (60,992) the two individual capacity factors k′ᵢ for both enantiomers was provided, and these data served for the affinity distribution analyses that required the individual k′ᵢ values.

Binding constant and free-energy computations. The interpretation of the chromatographic data in terms of ligand/stationary-phase association free-energies is based the theory of quantitative affinity chromatography [8, 9] which includes an equation of the form [10]
Kᵢ=Cᵢkᵢ (2)
where Cᵢ is the concentration of the stationary phase for a given selector compound in a specific column configuration and Kᵢ is the thermodynamic dissociation constant of the complex. Thus, a linear dependence exists between capacity factors and thermodynamic dissociation constants. However the values Cᵢ are variable, depending on column configuration and in general is not accurately known.

Free-energy differences, δΔG, associated with discrimination of enantiomers were calculated by use of an equation which applies to any separation i Eq. 2 [11]:
δΔG=−RTln(α) (3)

It may be seen that δΔG is independent of Cᵢ, because it cancels out based on Eqs. 1 and 2. Thus, the fact that Cᵢ values are not accurately known does not affect any of the analyses related to the distribution of α values.

Similarly, we evaluated the binding free-energies of the ligand/stationary phase associations by use of Eq. 4.
ΔGᵢ=−RTln(CᵢKᵢ). (4)
and employed methods for addressing the potential variance of Cᵢ values.

Both affinity and discrimination data were subjected to binning whereby normalized histograms were constructed describing the frequencies of the different free energy values or differences. This, and all other calculations (except where otherwise specified) were performed with MATLAB 6.5 (The MathWorks, Inc.).

Results

The distribution of enantio-discrimination parameters. We asked what might be the statistical distribution of enantiomer discrimination measures. To this end, we analyzed the entire set of data in the CHIRBASE compendium [6, 7], more than 70,000 chiral column separations. We extracted all elution separation factors, α and constructed a normalized binned frequency plot for all the a values (FIG. 1).

The enantiomer discrimination parameters spanned a range from 1.01 to 8.5, and showed a distinct functional distribution, whereby lower values were much more prevalent, while the highest values were very rare. As an example, 90% of all α values were lower than 2.2, while only 1% were higher than 5.0. Intriguingly, the chiral discrimination factors were found to obey a distinct power law P(α)=α^{-x}, with a best fit value of x=3.76.

In the past, chiral discrimination was reported to depend on the molecular size of the selector, whereby larger molecules exert higher α values [3]. To test whether this holds true for the very large dataset analyzed here, we recalculated the frequency distribution for triads, sorted according to the molecular weights of the chiral stationary phases (FIG. 2). Surprisingly, the P(α) distribution was found to be practically independent of molecular size of the selector up to a size of 600 Dalton.

Previous studies have shown that ligand-receptor affinities are governed by binomial or Poisson probability distributions [12-14]. However, attempts to fit P(α) to such functional dependences resulted in a much poorer fit (FIG. 1). This result is significant, because it reveals that the underlying law that may govern chiral selectivity is inherently different from that which directs the binding interactions themselves.

While P(α) failed to conform to a Poisson distribution, we reckoned that the probability distribution for the individual capacity factors kᵢ should. This is because these factors constitute bona fide binding constants, governed by the free energy of interaction between selector and selectand. Furthermore, we realized that such affinity data constitute one of the largest compendia of its kind, hence can provide additional support for the receptor affinity distribution model [13, 14] However, k′ᵢ values are not true equilibrium constants, but are related to them as k′ᵢ=Kᵢ/Cᵢ (Eq. 2). In order to examine the effect of the perturbation by the independent distribution of Cᵢ, we plotted the distribution P(k′ₘ) for compounds whose enantiomers correspond to a defined range of values k′₀<k′ₙ<k′₁ where m and n denote members of an enantiomer pair (FIG. 3). The resultant conditional exponentially declining distribution for RTln(kᵢ), showed that the exponent b (Eq. 7, appendix) has roughly the same value as for the overall P(δΔG), indicating that the distribution of the Cᵢ values has a relatively minor contribution, i.e. a smaller variance.

Subsequently, we selected at random one of each pair of kᵢ values, and computed the corresponding probability distribution, P(Kᵢ) for the entire dataset. The data were found to nicely obey a Poisson distribution for several values of Cᵢ (FIG. 4). Furthermore, the actual best fit parameters for the CHIRBASE dataset were rewardingly found to be in good agreement with previous analyses [14]. In particular, the new data were able to predict by extrapolation a set of independently measured frequencies of high affinity interactions taken from experiments in the realm of combinatorial chemistry ([14] and references thereof).

Interpreting P(α) through affinity distributions. In an attempt to interpret the above results we address the discrimination values as differences between values sampled from an underlying affinity distribution. Because of the significant result that the affinity values for individual enantiomers towards their selector obeys a Poisson-RAD distribution, we examined the distribution that would emerge based on the null hypothesis that k′1 and k′2 for every compound are completely uncorrelated. For this, random pairs were drawn out of the collection of experimental k′i values, and the probability distribution P(k′ᵢ/k′ⱼ) was drawn (FIG. 1, broken line). Obviously, the resultant curve, which constitutes a difference Poisson dependence, deviates strongly from that which represents the observed P(α) distribution. This discrepancy is not unexpected, however, because there are previous indications that such correlation exists. Indeed, plotting a correlation diagram for k′ᵢ values for all enantiomer pairs (FIG. 5) shows a correlation coefficient of 0.83, a value that seems to underlie the actual P(α) distribution. Such a value is necessary, although not sufficient for generating the observed statistical behavior, as this behavior will also depend on the specific set of conditional affinity distributions defined by the enantiomer/selector interaction.

A string complementarity model of enantio-selection.

While a Poisson distribution does not seem to explain the behavior of P(α), we examined the possibility that a model may be constructed that will rationalize the distribution of α values based on the distribution of the underlying kᵢ values. Consider an L-shaped string of length B as a model for an asymmetric selector (FIG. 6). The two antipodal selectands are represented by similar strings of length B which are mirror images of each other, but not super imposable in two dimensions. This model merges features of previously explored string-based models for molecular recognition, with concepts stemming from the three point attachment model for chiral discrimination. Thus, the two attachment points to the selector that preserve their interaction in both selectand enantiomers are represented by a long arm of length M, delimited by the chiral center. The third attachment point, which can form an interaction only in one of the isomers is represented by the shorter, B-M long arm. This model harbors a quantitative manifestation of the partial correlation between the binding energies of the two enantiomers: M elementary interactions are identical between the antipodes, while another B-M such interactions are only present in one of the isomers. The free energy of binding is computed by a string complemetarity rule [15, 16], employing a complete geometrical overlap for one of the isomers, and overlap restricted to the long arm for the other isomer. String digits are modeled as Bernoulli random variables obtaining 1 with a probability p and 0 with a probability 1−p. The free energy of binding is computed by eq. 2. This allows us to generate a large number of δΔG values in a Monte Carlo simulation, using a set of three parameters. From these, a P(δΔG) function is computed, and an iterative fit procedure subsequently employed to obtain the parameters showing best fit to the experimental points. FIG. 7 shows that the model adequately predicts the functional dependence of measured P(δΔG) data.

Discussion

Simple phenomenological models based on string complementarity have proven effective in predicting the functional form of affinity distributions [13-17]. We therefore attempted to develop an analogous approach for predicting the conditional probability phenomena that underlie enantio-selection.

It is possible to produce a more elaborate model in several ways: a) a three-dimensional, four-point attachment model [18] may be conceived. b) the model considered here is based on the assumption of no free energy contribution by the molecular aspect representing the third arm. An alternative that could be considered is that in some cases such moiety could generate repulsion and contribute a positive, inhibitory free energy of interaction. In general, models for enantio-selection are often based on simplifying assumptions, aimed at capturing a principle that will account for some quantitative aspects of the data. Following this example, and based on the notion of parsimony, we present the simplest model that shows an acceptable capacity to account for the experimental data.

In relation to this, it is worth mentioning a contradictory observation in which D'Souza et. al. [19] reports a chiral discrimination of haptens through exclusive binding of antibody to the OCD of the hapten enantiomers.

One of the surprising outcomes of this study is the discrepancy between the functional forms of distribution of binding free-energies and that of enantiomer discriminations. This discrepancy was shown to be the result of a particular correlation between the binding affinities of the enantiomers suggesting that a) both enantiomers bind to the same structural location on the selector molecule and b) enantio-selectivity results from slight differences in the mode of binding of the two enantiomers.

It is a well known fact that enantio-discriminating free energy differences are typically lower in absolute value than the binding energies experienced between the separated ligands and the stationary phase. Lipkowitz [23] reports that in many cases the binding energies exceed the differentiating energies by up to 2-3 orders of magnitude.

We have employed tools from quantitative affinity chromatography for the construction of distributions of separation and binding free energies in chiral elutions. Surprisingly a distinction was revealed between the functional forms of these two distributions suggesting that different mechanisms underlie the two processes.

The approach of employing quantitative affinity chromatography for the study of affinity distributions has been previously employed by Inman & Barnett [20, 21] where 85 different small ligands were tested against an IgG1 monoclonal antibody specific for 2,4-dinitrophenyl hapten. In a more recent reanalysis of Inman's dataset it was shown that the data also agrees with the RAD model developed in our lab. More so, it intriguingly suggested that a single universal function, ψ, may describe the affinity distributions from various experimental sources and that this distribution may have predictive capacity. In attempt to asses these predictions we assumed that ψ is functionally close in shape to the our measured fitted Poisson affinity distribution and tested the ability of this function to describe different types of affinity data statistics.

Specifically, we ask whether a Poisson ψ may describe the probability of obtaining the affinity value obtained from screening a phage display library of a given size. As in Rosewald et. al. frequencies for the phage display affinity values were estimated by the inverse of the library size from which they were extracted. Results shown in FIG. 4 demonstrate that without further free-parameter optimization, ψ predicts frequencies phage display products even though these are over an order of magnitude higher in affinity than those for which it was fit.

Here we further extended this approach asking of the distribution of chiral separation factors describing the ration of the affinities of the two enantiomers.

We are aware that these observations (size independence) are in disagreement with theoretical predictions [3, 15, 17] but state that this may simply be the outcome of the low complexity of discriminating molecules in the CHIRBASE.

Also, it serves as an indication that in the majority of cases the most enantio-discriminating region overlaps that of the highest affinity interaction with the separated ligands. This question of enantio-discriminating vs. binding domains in chiral selectors has been previously discussed [22] in the context of cyclodextran based chiral stationary phases.

### LEGEND TO FIGURES

FIG. 1:

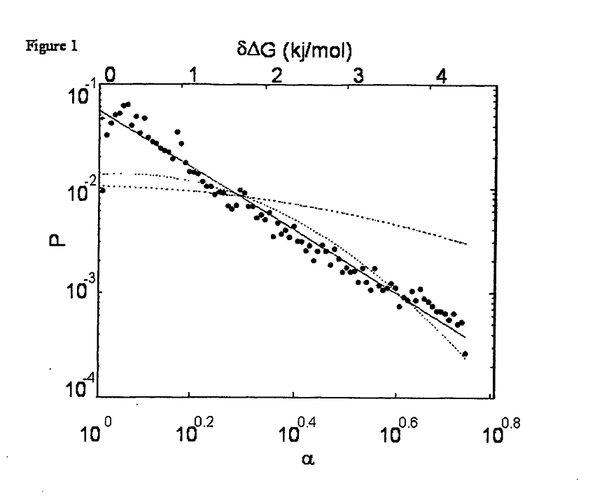

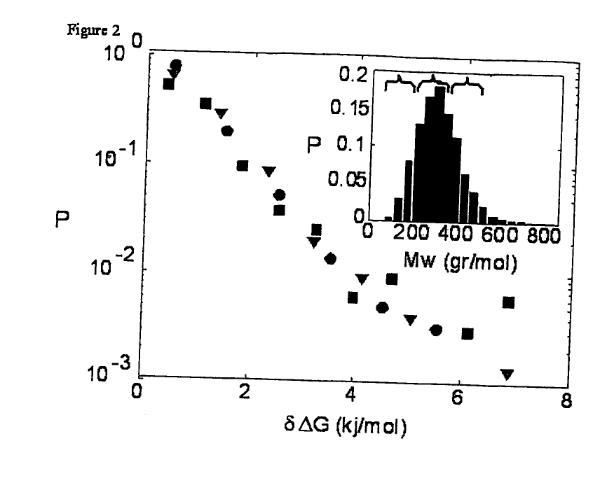

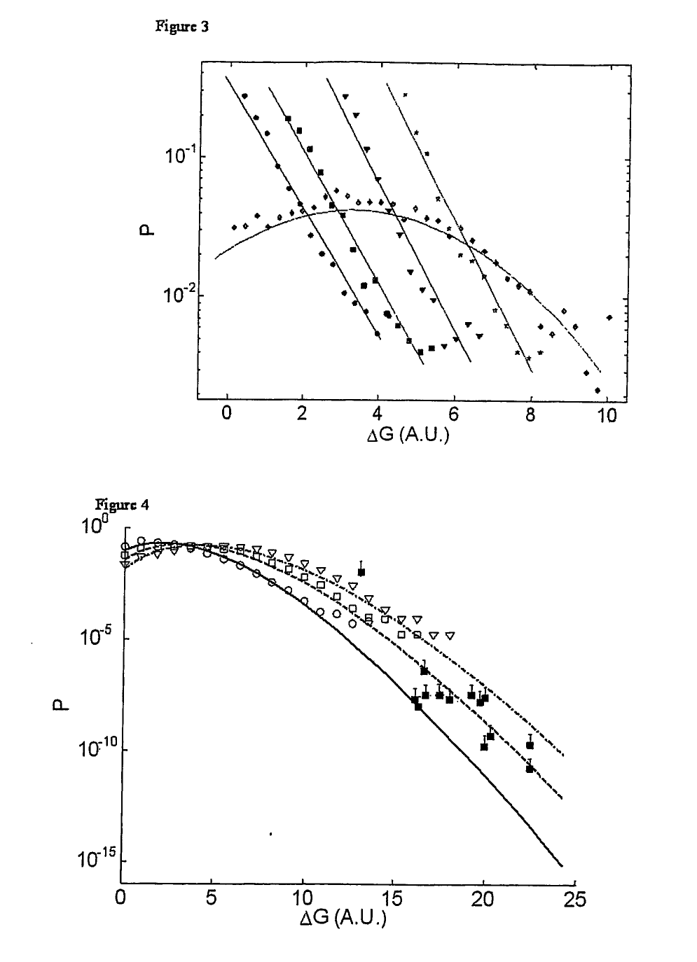

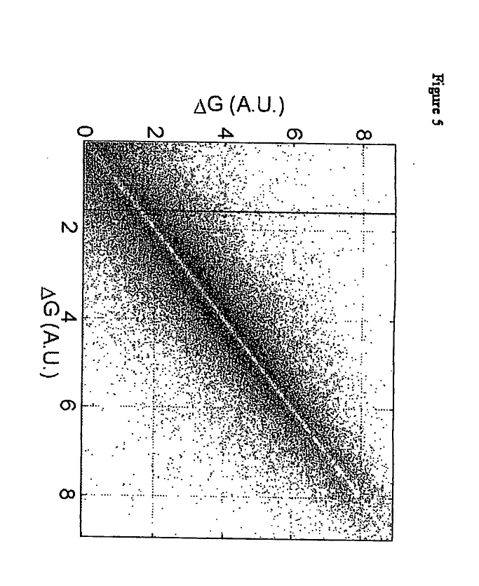

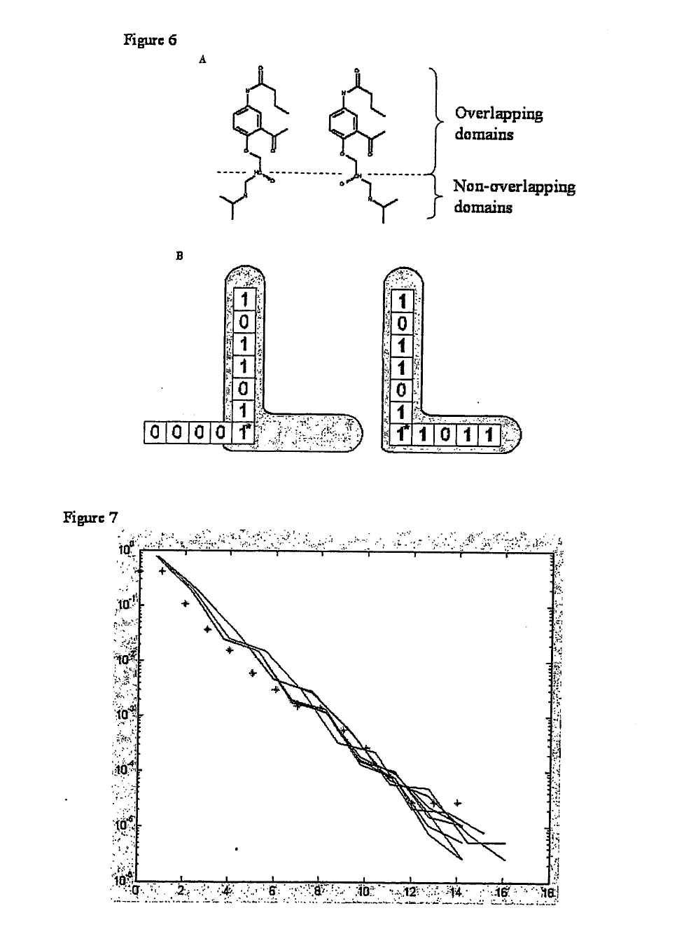

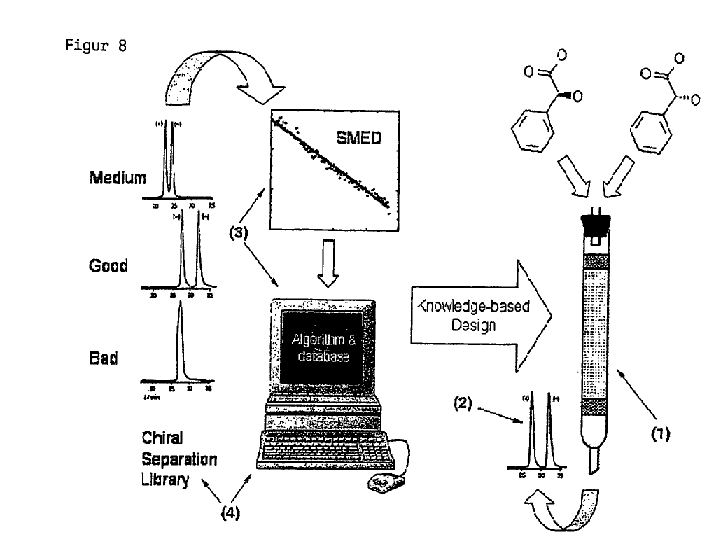

Frequency histogram for enantiomer discrimination free-energy differences, E, fit with exponential (red) and Gaussian (purple) distributions. The free-parameters obtained through the nonlinear fit optimizations are λ=0.218 for the exponential distribution and μ=3.0, σ=0.74 for the Gaussian distribution. Also plotted (blue) is a distribution of enantiomer discrimination free-energy differences calculated from the fit RAD assuming no correlation between the binding affinities of enantiomer related CSP/ligand complexes. The plots were created through the binning of 37,707 data values into 149 bins.

FIG. 2:

The molecular weight distribution of chiral stationary phases (inset) and the dependence of the distribution, g, of enantiomer discriminating free-energy differences, E, on the molecular weight of the chiral selector. Shown are the frequency histograms of enantiomer discrimination free energy differences calculated from separations performed with CSPs of sizes 0-200 gr/mol (red), 200-400 gr/mol (blue) and 400-600 gr/mol (black). The plot was calculated from of data set of 60,992.

FIG. 3:

Binding free-energy distribution of ligand/selector pairs (∘) fit with a RAD (--). Also shown are the maximal affinity scores (translated into free-energy values) of phage display screen products plotted against their inverse library size (▮). The extrapolated RAD with its free-parameters optimized to best fit the CHIRBASE affinity data (λ=4.64, σ=0.9 kj/mol) is shown to predict frequency values of phage display maximal affinities. Calculation of affinity values (ΔG) was performed through Eq. 4 and is thus constrained to the errors resulting from neglecting the particular column specifications. In order to sufficiently represent both high and low affinity data, the data fitting procedure was performed in the semi-logarithmic scale (Log P vs ΔG). Temperatures for the calculation of free-energy values were taken from CHIRBASE where it was available and assumed to be room temperature (25C) otherwise. The majority of the affinity values describing ligand/stationary phase interactions were observed around Ka=2.65 corresponding to the binding free-energy of 2.5 kj/mol and only a few (˜5%) reach affinities above Ka=18.5 (7 kj/mol). In accordance with the suggestion of the universality of the RAD Code: BindingEnergy137.m

FIG. 4:

Correlation of the binding free-energies of the enantiomer related ligand/CSP complexes. The figure shows ΔG values of ligand/CSP interactions plotted against the values corresponding to the same CSP and the ligands enantiomer. The red lines illustrate the concept of the conditional affinity distribution by addressing the subset of elutions for which one of the enantiomer/stationary phase interactions for each elution is confined to a particular affinity interval. A reduced concentration of data points on the diagonal reflects the fact that only successful resolutions were included in the analysis.

FIG. 5:

Conditional affinity frequency-histograms calculated by grouping the chiral resolution data according to the AG value of ligand/CSP interactions. A. Frequency histograms were calculated for groups of ligand/CSP pairs maintaining the condition that the ligands enantiomer bind the stationary phase with an interaction characterized by AG values of 1 to 1.3 (red), 3 to 3.3 (blue) and 5 to 5.5 (purple). In order to verify the results we tested the conditional affinity/frequency data against a distribution of E values computed from the same sub-dataset (black). This means that a set of enantiomer pairs was constructed such that each pair contained an enantiomer binding the CSP with a value constrained to the preferred interval. From the values within this set we constructed the distribution of ΔG values and a distribution of E values. Also, we show a distribution of ΔG values for a randomly collected set of the same size (green).

FIG. 6:

Dependence of the conditional affinity distribution on the binding free-energy, x_{E}, between the CSP and one of the separated enantiomers. A. This dependence is quantified by the free parameters a and b defining the distribution through Eq. 7. Each point on the plot corresponds to the optimal values of a and b obtained by fitting Eq. 7 to the frequency histogram of ΔG values computed for the condition specified by x_{E}. We show linear least square fits to the x_{E} dependent free-parameter changes. B. From the fits shown in A an expression for the conditional affinity distribution function was derived and plotted for x_{E} values of 1 kj/mol (blue), 3 kj/mol (green), 6 kj/mol (red), 9 kj/mol (light blue) and 12 kj/mol (purple). A cutoff was employed to exclude samples containing less than 30 elution experiments.

FIG. 7:

A general model for the enantio-selectivity. While enantiomers are defined by their incapability of spatial overlap, it is still possible to overlap distinct enantiomer molecular domains defined by the locations of their asymmetric centers (A). Based on this we derive a model (B) by considering two enantiomeric molecular structures (red and blue outlined grids) and a chiral selector surface (gray). We conceptually divide the binding contour of the enantiomer into a grid and assign a probability that an area interval, DA, on the molecular surface will either remain in or deviate from the spatially overlapping domains of the enantiomers (the construction could be similarly extended to describe enantiomers with more than one asymmetric center). We show (see text) that such a construction leads to the observed Exponential distribution of enantiomer discrimination free-energy differences.

FIG. 8:

A sketch of an example for the experimental setup mentioned in the claims 3, 10-15. A device is set up which is able to separate (at least partially) the enantiomers (1) and subsequently measure the capacity factors which are related to the affinity values for both enantiomers (2). The measurement values are processed (3) and stored (4) in a processing unit (e.g. a computer) and afterwards compared to the already existing data in (4) to determine an estimation of the statistics. For claim 10 an additional database with 3D-structures is available in the processing unit.

### REFERENCES

1. Labrie, J., J. F. Mercier, and G. W. Slater, An exactly solvable Ogston model of gel electrophoresis. V. Attractive gel-analyte interactions and their effects on the Ferguson plot. Electrophoresis, 2000. 21(5): p. 823-33.
2. Sundaresan, V. and R. Abrol, Towards a general model for protein-substrate stereoselectivity. Protein Sci, 2002. 11(6): p. 1330-9.
3. Katzenelson, O. and D. Avnir, Quantitative chirality/enantioselectivity relations in large random supramolecular structures. Chemistry, 2000. 6(8): p. 1346-54.
4. Sips, R. J., On the structure of a catalyst surface. J. Chem. Phys., 1948. 16(5): p. 490-495.
5. Roussel, C., P. Piras, and I. Heitmann, An approach to discriminating 25 commercial chiral stationary phases from structural data sets extracted from a molecular database. Biomed Chromatogr, 1997. 11(5): p. 311-6.
6. Koppenhoefer, B., et al., CHIRBASE, a molecular database for the separation of enantiomers by chromatography. J Chromatogr A, 1994. 666(1-2): p. 557-63.
7. Koppenhoefer, B., et al., CHIRBASE, a graphical molecular database on the separation of enantiomers by liquid-, supercritical fluid-, and gas chromatography. Chirality, 1993. 5(4): p. 213-9.
8. Chaiken, I. M., Analytical affinity chromatography in studies of molecular recognition in biology: a review. J Chromatogr, 1986. 376: p. 11-32.
9. Winzor, D. J., Quantitative affinity chromatography. J Biochem Biophys Methods, 2001. 49(1-3): p. 99-121.
10. Chaiken, I. M., Analytical Affinity Ahromatography. CRC handbooks. 1987, Boca Raton, Fla.: CRC Press, 1987.
11. Ching, C. B., et al., Chromatographic resolution of the chiral isomers of several beta-blockers over cellulose tris(3,5-dimethylphenylcarbamate) chiral stationary phase. Chirality, 1992. 4(3): p. 174-7.
12. Goldstein, B., Theory of hapten binding to IgM: the question of repulsive interactions between binding sites. Biophys Chem, 1975. 3(4): p. 363-7.
13. Lancet, D., E. Sadovsky, and E. Seidemann, Probability model for molecular recognition in biological receptor repertoires: significance to the olfactory system. Proc Natl Acad Sci USA, 1993. 90(8): p. 3715-9.
14. Rosenwald, S., R. Kafri, and D. Lancet, Test of a statistical model for molecular recognition in biological repertoires. J Theor Biol, 2002. 216(3): p. 327-36.
15. Perelson, A. S. and G. Weisbuch, Immunology for physicists. Reviews of Modern Physics, 1997. 69(4): p. 1219-1267.
16. Lancet, D., A. Horovitz, and E. Katchalski-Katzir, Molecular recognition in biology: models for analysis of protein-ligand interactions, in The Lock-and-Key Principle, J. P. Behr, Editor. 1994, John Wiley & Sons Ltd. p. 25-71.
17. Perelson, A. S., Immune Network Theory. Immunological Reviews, 1989. 110: p. 5-36.
18. Mesecar, A. D. and D. E. Koshland, Sites of binding and orientation in a four-location model for protein stereospecificity. Iubmb Life, 2000. 49(5): p. 457-466.
19. D'Souza, L. J., et al., Remarkable remote chiral recognition in a reaction mediated by a catalytic antibody. J Am Chem Soc, 2002. 124(10): p. 2114-5.
20. Inman, J. K., The Antibody combinig region: Speculations on the hypothesis of general multispecificity, in Theoretical Immunology, P. A. S. Bell G. I., Pimbly G. H. Jr., Editor. 1978, Marcell Dekker: New York.
21. Inman, J. K., A study of multispeci.c interactions by quantative affinity chromatography. In: Affinity Chromatography and Biological Recognition(Chaiken, I. M., Wilcheck, M. Parkih, I., eds), Orlando, Academic Press. 1983.
22. Lipkowitz, K. B., R. Coner, and M. A. Peterson, Locating regions of maximum chiral discrimination: A computational study of enantioselection on a popular chiral stationary phase used in chromatography. Journal of the American Chemical Society, 1997. 119(46): p. 11269-11276.
23. Lipkowitz, K. B. Acc Chem Res 33, 555-62, 2000.
24. Maier, N. M., Franco, P. and Lindner, W., J. of Chromatography A, 906(1-2), 3-33, 2001.
25. Lough, W. J. Ed., (1989) “Chiral liquid chromatography”, Blackie, Glasgow, IX+276 pp., ISBN 0-216-92499-5, 1989.

1. A method of identifying molecules having enantio-discriminating regions comprising applying the chiral probability law disclosed herein to a plurality of molecules thereby identifying the molecules having enantio-discriminating regions.

2. A method of uncovering a chiral selector of a specific enantiomer according to claim 1 comprising, obtaining a three dimensional structure of at least a unique portion of the specific enantiomer and applying the chiral probability law disclosed herein to a plurality of molecules and said at least a unique portion of the specific enantiomer to thereby uncover the chiral selector of a specific enantiomer.

3. A system using the method according to claim 1 for identifying molecules having enantio-discriminating regions the system comprising a processing unit being designed and configured to apply the chiral probability law disclosed herein to a plurality of molecules.

4. Use of the chiral probability law disclosed herein to a plurality of molecules to design experiments by giving a statistical significant measure derived from a small sample set on how many compounds will have to be tested at least to obtain a predefined minimal number of compounds
a) exceeding a certain threshold for the enantio-discriminating value alpha, or
b) being within a certain range for the enantio-discriminating value alpha.

5. Use of the chiral probability law disclosed herein to a plurality of molecules to design tailored chemical libraries, supported by measurement results about the enantio-discriminating of a small subsample of the library to obtain a predefined minimal number of compounds
a) exceeding a certain threshold for the enantio-discriminating value alpha, or
b) being within a certain range for the enantio-discriminating value alpha.

6. Use according to claim 4, wherein the predefined minimal number of components is obtained within a subset of a library.

7. Use of the method of claim 5, wherein the tailored chemical libraries are tailored subtypes of chemical libraries.

8. Use of the chiral probability law disclosed herein to a plurality of molecules for selecting the best suitable libraries according to claim 4 for a given profile.

9. Use according to claim 4, whereby at least part of the 3D-structure of either a selector or a selectand are incorporated.

10. The system according to claim 3, including, use of the chiral probability law disclosed herein to a plurality of molecules to design experiments by giving a statistical significant measure derived from a small sample set on how many compounds will have to be tested at least to obtain a predefined minimal number of compounds
a) exceeding a certain threshold for the enantio-discriminating value alpha, or
b) being within a certain range for the enantio-discriminating value alpha;
and whereby the analyzing methods are carried out on the processing unit applied to the observed statistics.

11. The system according to claim 3, including, use of the chiral probability law disclosed herein to a plurality of molecules to design tailored chemical libraries, supported by measurement results about the enantio-discriminating of a small subsample of the library to obtain a predefined minimal number of compounds
a) exceeding a certain threshold for the enantio-discriminating value alpha, or
b) being within a certain range for the enantio-discriminating value alpha;
and whereby the analyzing methods are carried out on the possessing unit applied to the observed statistics.

12. The system according to claim 3, including, use according to claim 4, wherein the predefined minimal number of components is obtained within a subset of a library and whereby the analyzing methods are carried out on the possessing unit applied to the observed statistics.

13. The system according to claim 3, including, use of the method of claim 5, wherein the tailored chemical libraries are tailored subtypes of chemical libraries and whereby the analyzing methods are carried out on the possessing unit applied to the observed statistics.

14. The system according to claim 3, including, use of the chiral probability law disclosed herein to a plurality of molecules for selecting the best suitable libraries according to claim 4 for a given profile and whereby the analyzing methods are carried out on the possessing unit applied to the observed statistics.

15. The system according to claim 3, including, use according to claim 4, whereby at least part of the 3D-structure of either a selector or a selectand are incorporated and whereby the analyzing methods are carried out on the possessing unit applied to the observed statistics.
